(19) United States
(12) Patent Application Publication
(10) Pub. No.: US 2013/0226522 A1
(43) Pub. Date: Aug. 29, 2013

(54) RUBBER PRODUCT ELASTIC RESPONSE PERFORMANCE PREDICTION METHOD, DESIGN METHOD, AND ELASTIC RESPONSE PERFORMANCE PREDICTION APPARATUS

(71) Applicant: Keizo Akutagawa, Kodaira-shi (JP)

(72) Inventor: Keizo Akutagawa, Kodaira-shi (JP)

(73) Assignee: BRIDGESTONE CORPORATION, Chuo-ku, Tokyo (JP)

(21) Appl. No.: 13/877,592

(22) PCT Filed: Oct. 4, 2011
(86) PCT No.: PCT/JP2011/072886

§ 371 Date: May 16, 2013

(30) Foreign Application Priority Data
Oct. 5, 2010 (JP) 2010-225984

(51) Int. Cl. G01N 33/44

(52) U.S. Cl. CPC G01N 33/445
USPC 702/182

(57) ABSTRACT

An elastic response performance prediction method that employs a finite element analysis method to predict an elastic response performance expressing deformation behavior of a rubber product. The elastic response performance of the rubber product is predicted by employing a constitutive equation that expresses temperature and strain dependence of strain energy in the rubber product, and that incorporates a number of links between cross-linked points in a statistical molecule chain, which is expressed using a parameter representing extension crystallization.

### TECHNICAL FIELD

The present invention relates to an elastic response performance prediction method and design method for a rubber product, and to an elastic response performance prediction apparatus. In particular the present invention relates to a prediction method that employs a Finite Element Analysis (FEA) method to predict an elastic response performance of a rubber product, to a rubber product design method employing the prediction method, and to an elastic response performance prediction apparatus thereof.

### BACKGROUND ART

When designing a rubber product, methods that employ 3-D Finite Element Analysis (FEA) methods to predict the elastic response thereof, and that employ such analysis and simulation results have already been implemented for tens of years. Energy constitutive equations that are employed in FEA computation and in the analysis reflect a stress-strain relationship of a rubber material, migrate linear elasticity equations into Mooney-Rivlin equations, and recently non-linear constitutive equations are being introduced for regions of large deformation in such energy constitutive equations.

Moreover, recently with regard to such a rubber material constitutive equation, under the principles of lattice deformation theory developed with molecular statistical thermodynamics based on the extension of rubber molecular chains, there is a proposal for a constitutive equation that is capable of expressing temperature dependence, an important factor in the design of a lot of industrial rubber materials, such as tires, using a parameter with a physical meaning (Japanese Patent Publication 4299735).

A summary of such a constitutive equation follows.

First, tensional stress in rubber is expressed by a shear elastic modulus G and a tension direction extension ratio λ using the following Equation (1).

$$σ = 2 G ( λ - \frac{1}{{λ}^{2}} ) \qquad ( 1 )$$

Moreover, the shear elastic modulus G of rubber is computable, as shown in the following Equation (2), by differentiating the Helmholz free energy A of statistical thermodynamics with respect to I₁.

$$\frac{G}{2} = \frac{\partial A}{\partial {I}_{1}} \qquad ( 2 )$$

The average energyĀ_{α} of a system in a thermodynamic equilibrium state is expressed by the following Equation (3).

$${\overline{A}}_{α} = \frac{1}{Z} \sum {A}_{α} {e}^{- β {A}_{α}} = - {( \frac{\partial log Z}{\partial β} )}_{\{ Γ \}} \qquad ( 3 )$$

β here is equivalent to 1/(kΔT), wherein k represents the Helmholz constant and ΔT represents the difference from a polymer glass transition temperature T_{g} to the measured temperature T. Moreover, A_{α} represents the energy level. Z is a distribution function for standardizing the energy of the system, and is expressed by the following Equation (4).

$$Z = {\sum }_{α} {e}^{- β {A}_{α}} = {\sum }_{α} {e}^{- β ( {U}_{α} - {TS}_{α} )} \qquad ( 4 )$$

Here, as expressed in the following Equation (5), U_{α} can be assumed to be equivalent to Hamiltonian H from a statistical thermodynamic perspective.

U_{α}=H(Temp, Constraints) (5)

Wherein, the Hamiltonian H is expressed as a function of temperature conditions and constraint conditions defining microstates in statistical thermodynamics.

Two states are set, a low temperature state and a high temperature state, as a model expressing temperature dependence of rubber molecules, and state numbers of rubber molecule called r₁ and r₂ are distributed in strain energy fields called I₁ₐ and I_{1b}. This can be represented by the following Equation (6) when this is applied to express all of the states as a function.

$$\begin{matrix}Z = {\sum }_{∝} {e}^{- {βλ}_{∝}} \\ = {\sum }_{α} {e}^{- β ( {U}_{α} {TS}_{α} ) \cdot } \\ = {e}^{β TS} ( {e}^{- \frac{- {l}_{1 a} - {I}_{1 b} - κ}{k Δ T}} + {e}^{- \frac{- {I}_{1 a} + {I}_{1 b}}{k Δ T}} + {e}^{- \frac{{I}_{1 a} - {I}_{1 b}}{k Δ T}} + {e}^{- \frac{{I}_{1 a} + {I}_{1 b} - κ}{k Δ T}} ) \\ = 2 {e}^{β TS} [ {e}^{\frac{κ}{k Δ T}} cosh ( \frac{{I}_{1 a} + {I}_{1 b}}{k Δ T} ) + cosh ( \frac{{I}_{1 a} - {I}_{1 b}}{k Δ T} ) ]\end{matrix} \qquad ( 6 )$$

Wherein κ represents molecular recoil energy. Two states of different rubber molecule energy states are represented by r₁=−1, r₂=1, and the statistical thermodynamic average thereof is represented by <r₁·r₂>. This product expresses the two extreme end energy states, and when this product is +1 this represents rubber molecules being in the same state, and when this product is −1 this represents rubber molecules being in different states. Assuming that the strain energy field contribution is equal for all of the molecules, then by making I₁=I₁ₐ=I_{1b}, Equation (6) above can be expressed by the following Equation (7).

$$\begin{matrix}Z = {\sum }_{α} {e}^{- β {A}_{α}} \\ = 2 {e}^{β TS} [ {e}^{\frac{κ}{k Δ T}} cosh ( 2 β \cdot {I}_{1} ) + 1 ]\end{matrix} \qquad ( 7 )$$

Substituting Equation (7) in above Equation (3) expresses the rubber elastic modulus G by the following Equation (9).

$$\begin{matrix}{\overline{A}}_{α} = \frac{1}{Z} \sum {A}_{α} {e}^{- β {A}_{α}} \\ = - {( \frac{\partial log Z}{\partial β} )}_{\{ Γ \}} \\ = - \frac{\partial }{\partial β} [ {e}^{\frac{κ}{k Δ T}} cosh ( 2 β \cdot {I}_{1} ) + 1 ] - T \cdot S\end{matrix} \\ \begin{matrix}\frac{G}{2} = \frac{\partial {\overline{A}}_{α}}{\partial {I}_{1}} \\ = \frac{\partial U}{\partial {I}_{1}} - T \cdot \frac{\partial S}{\partial {I}_{1}} \\ = - \frac{\partial }{\partial β \partial {I}_{1}} [ {e}^{\frac{κ}{k Δ T}} cosh ( 2 β \cdot {I}_{1} ) + 1 ] - T \cdot \frac{\partial S}{\partial {I}_{1}}\end{matrix} \qquad ( 9 )$$

Considering a stretched molecule, the second term of the entropy term in Equation (9) is expressed using a Langevin function by the following Equation (10).

$$T \cdot \frac{\partial S}{\partial {I}_{1}} = - vRT [ \frac{1}{2} + \frac{3}{50 n} ( 3 {I}_{1} - \frac{2}{λ} ) + \frac{297}{6125 {n}^{2}} ( 5 {I}_{1}^{2} - 4 {I}_{2} - 4 \frac{{I}_{1}}{λ} ) ] \qquad ( 10 )$$

Wherein n is defined as the number of links of a statistical molecule chain between cross-linked points.

Accordingly, Japanese Patent No. 4299735 proposes a constitutive equation that is capable of representing temperature dependence and employs a parameter with physical meaning.

Moreover, as illustrated in FIG. 6, a strain energy function (solid line) disclosed in Japanese Patent No. 4299735 is able to replicate test values (plots) over a wide range of extension ratios and temperature regions.

### DISCLOSURE OF INVENTION

### Technical Problem

However, in the strain energy function disclosed in Japanese Patent Publication 4299735, since consideration is not given to extension crystallization, this leads to the issue that the applicable range thereof is limited to rubbers without extension crystallization properties, such as styrene butadiene rubber.

The present invention is directed towards solving the above issue, and an object thereof is to provide an elastic response performance prediction method, a rubber product design method and an elastic response performance prediction apparatus that are capable of raising prediction precision of the elastic response performance of rubber products even when the rubber product has extension crystallization properties.

### Solution to Problem

In order to achieve the above objective, a first aspect of the present invention is an elastic response performance prediction method that predicts an elastic response performance expressing deformation behavior of a rubber product by predicting the elastic response performance of the rubber product by employing a constitutive equation that expresses temperature and strain dependence of strain energy in the rubber product, and that incorporates a number of links between cross-linked points in a statistical molecule chain which is expressed using a parameter representing extension crystallization.

A second aspect of the present invention is an elastic response performance prediction apparatus that predicts elastic response performance expressing deformation behavior of a rubber product by predicting the elastic response performance of the rubber product by employing a constitutive equation that expresses temperature and strain dependence of strain energy in the rubber product, and that incorporates a number of links between cross-linked points in a statistical molecule chain which is expressed using a parameter representing extension crystallization.

According to the first aspect and the second aspect of the present invention, when a filler based rubber that exhibits extension crystallization properties is observed at the micro-observation level, in a diffuse deformation region of a rubber product, a constitutive equation is employed that expresses temperature and strain dependence of strain energy, and that incorporates the number of links between the cross-linked points in the statistical molecule chain which is expressed using the parameter representing extension crystallization. Accordingly, elastic response performance of a rubber product can be predicted with good precision even when the rubber product exhibits extension crystallization properties.

According to a third aspect of the present invention, a number of links n between the cross-linked points in the statistical molecule chain is expressed by the following Equation (I):

n=α·(1−X_{c})·exp(−ε·β) (I)

Wherein α represents a frequency factor of statistical segment motion, and ε represents an activation energy of statistical segment motion. β=1/RT_{g} wherein R is a gas constant and T_{g} is a glass transition temperature X_{c} represents a crystallization ratio as a parameter expressing the extension crystallization, and X_{c} is expressed by the following Equation (II) when a material of the rubber product exhibits extension crystallization properties:

$${X}_{c} = ( \frac{{U}_{1} - {U}_{0}}{Δ {H}_{0}} ) = ( \frac{Δ U}{Δ {H}_{0}} ) \qquad ( II )$$

Wherein U₀ represents internal energy in a non-deformed state, U₁ represents internal energy in a deformed state, and ΔH₀ represents entropy of solution when crystals melt.

According to a fourth aspect of the present invention, the crystallization ratio X_{c} may be set at 0 when the material of the rubber product does not exhibit extension crystallization properties.

According to a fifth aspect of the present invention, the constitutive equation is the following Equation (III):

ΔA=(U₁−TS₁)+p(V₁−V₀)−(U₀−TS₀) (III)

Wherein A represents Helmholz free energy, U₀ represents internal energy in a non-deformed state and U₁ represents internal energy in a deformed state. p represents pressure, V₀ represents volume in a non-deformed state and V₁ represents volume in a deformed state. T represents absolute temperature, S₀ represents entropy in a non-deformed state, and S₁ represents entropy in a deformed state. Each of the terms of Equation (III) are expressed by the following Equations (IV) to (VI):

$$\begin{matrix}{U}_{1} - T \cdot {S}_{1} = \frac{{e}^{{β}^{′} \cdot κ} \{ κ cosh ( 2 {β}^{′} ( {I}_{1}^{′} - 3 ) ) + 2 ( {I}_{1}^{′} - 3 ) \cdot sinh ( 2 {β}^{′} ( {I}_{1}^{′} - 3 ) ) \}}{{e}^{{β}^{′} \cdot κ} cosh ( 2 {β}^{′} ( {I}_{1}^{′} - 3 ) ) + 1} - \frac{N}{β} \cdot \{ \frac{1}{2} {I}_{1} + \frac{3}{100 n} ( 3 {I}_{1}^{2} - 4 {I}_{2} ) + \frac{99}{12250 n} ( 5 {I}_{1}^{3} - 12 {I}_{1} {I}_{2} ) \} & ( IV ) \\ \begin{matrix}p ( {V}_{1} - {V}_{0} ) = B \cdot ( {V}_{1} - {V}_{0} ) \\ = {B ( {I}_{3}^{\frac{1}{2}} - 1 )}^{2} - \frac{1}{{β}^{′}} \{ \begin{matrix}ln [ 1 + {e}^{{β}^{′} \cdot κ} cosh ( 2 {β}^{′} ( {I}_{1}^{′} - 3 ) ) ] - \\ ln [ 1 + {e}^{{β}^{′} \cdot κ} ]\end{matrix} \}\end{matrix} & ( V ) \\ {U}_{0} - T \cdot {S}_{0} = \frac{κ \cdot {e}^{{β}^{′} \cdot κ}}{{e}^{{β}^{′} \cdot κ} + 1} + \frac{N}{β} ( \frac{3}{2} + \frac{45}{100 n} + \frac{2673}{12250 {n}^{2}} ) & ( VI )\end{matrix}$$

Wherein: I₁, I₂, and I₃ are expressed as functions of three extension ratios of deformation λ₁, λ₂, and λ₃ in xyz directions in three dimensional axes of rubber by I₁=λ₁²+λ₂²+λ₃², I₂=λ₁²·λ₂²+λ₂²·λ₃²+λ₃²·λ₁², and I₃=λ₁²·λ₂²·λ₃². n represents a number of links between the cross-linked points in the statistical molecule chain and κ expresses an intermolecular interaction energy coefficient. β=1/RT and β′=1/R(T−T_{g}) wherein R is a gas constant and T_{g} is a glass transition temperature, and I₁′ is expressed using a local interaction function λ_{micro} as a parameter expressing the intermolecular interaction by the following Equation (VII):

I₁′=λ_{micro}²(λ₁²+λ₂²+λ₃²)=λ_{micro}²·I₁ (VII)

A sixth aspect of the present invention is an elastic response performance prediction method that predicts elastic response performance expressing deformation behavior of a rubber product by predicting the elastic response performance of the rubber product by employing a constitutive equation that expresses temperature and strain dependence of an elastic modulus of the rubber product, and that incorporates a number of links between cross-linked points in a statistical molecule chain which is expressed using a parameter representing extension crystallization.

A seventh aspect of the present invention is an elastic response performance prediction apparatus that predicts elastic response performance expressing deformation behavior of a rubber product by predicting the elastic response performance of the rubber product by employing a constitutive equation that expresses temperature and strain dependence of an elastic modulus of the rubber product, and that incorporates a number of links between cross-linked points in a statistical molecule chain which is expressed using a parameter representing extension crystallization.

According to the sixth aspect and the seventh aspect of the present invention, when a filler based rubber that exhibits extension crystallization properties is observed at the micro-observation level, in a diffuse deformation region of a rubber product, a constitutive equation is employed that expresses temperature and strain dependence of an elastic modulus, and that incorporates the number of links between the cross-linked points in the statistical molecule chain which is expressed using the parameter representing extension crystallization. Accordingly, elastic response performance of a rubber product can be predicted with good precision even when the rubber product exhibits extension crystallization properties.

According to an eighth aspect of the present invention, a number of links n between the cross-linked points in the statistical molecule chain is expressed by following the Equation (VIII):

n=α·(1−X_{c})·exp(−ε·β) (VIII)

Wherein α represents a frequency factor of statistical segment motion, and ε represents an activation energy of statistical segment motion. β=1/RT_{g} wherein R is a gas constant and T_{g} is a glass transition temperature. X_{c} represents a crystallization ratio as a parameter expressing the extension crystallization, and X_{c} is expressed by the following Equation (IX) when a material of the rubber product exhibits extension crystallization properties:

$$\begin{matrix}{X}_{c} = ( \frac{{U}_{1} - {U}_{0}}{Δ {H}_{0}} ) \\ = ( \frac{Δ U}{Δ {H}_{0}} )\end{matrix} \qquad ( IX )$$

Wherein U₀ represents internal energy in a non-deformed state and U₁ represents internal energy in a deformed state, and ΔH₀ represents entropy of solution when crystals melt.

In a ninth aspect of the present invention the crystallization ratio X_{c} may be set at 0 when the material of the rubber product does not exhibit extension crystallization properties.

In a tenth aspect of the present invention the constitutive equation is the following Equation (X):

$$\begin{matrix}G = \frac{\partial W}{\partial {I}_{1}} \\ = \frac{\partial A}{\partial {I}_{1}} \\ = \frac{\partial U}{\partial {I}_{1}} - T \cdot \frac{\partial S}{\partial {I}_{1}}\end{matrix} \qquad ( X )$$

Wherein G represents a shear elastic modulus, W represents a strain energy coefficient and A represents Helmholz free energy. U represents internal energy, T represents absolute temperature and S represents entropy. I₁ is expressed as a function of three extension ratios of deformation λ₁, λ₂ and λ₃ in xyz directions in three dimensional axes of rubber by I₁=λ₁²+λ₂²+λ₃². Each of the terms of Equation (X) is respectively expressed by the following Equation (XI), Equation (XII), and Equation (XIII):

$$\begin{matrix}\frac{\partial U}{\partial {I}_{1}} = \frac{\begin{matrix} \\ - {e}^{{β}^{′} κ} \{ \begin{matrix}\begin{matrix}2 {e}^{{β}^{′} κ} sinh ( 2 {β}^{′} ( {I}_{1}^{′} - 3 ) ) cosh ( 2 {β}^{′} ( {I}_{1}^{′} - 3 ) ) + \\ 2 ( {β}^{′} κ + 1 ) sinh ( 2 {β}^{′} ( {I}_{1}^{′} - 3 ) ) +\end{matrix} \\ 4 {β}^{′} ( {I}_{1}^{′} - 3 ) cosh ( 2 {β}^{′} ( {I}_{1}^{′} - 3 ) ) + \\ 4 {β}^{′} ( {I}_{1}^{′} - 3 ) {e}^{{β}^{′} κ}\end{matrix} \}\end{matrix}}{{\{ {e}^{{β}^{′} κ} \cdot cosh ( 2 {β}^{′} ( {I}_{1}^{′} - 3 ) ) + 1 \}}^{2}} & ( XI ) \\ \frac{\partial S}{\partial {I}_{1}} = - vR [ \frac{1}{2} + \frac{3}{50 n} ( 3 {I}_{1} - \frac{2}{λ} ) + \frac{297}{6125 {n}^{2}} ( 5 {I}_{1}^{2} - 4 {I}_{2} - 4 \frac{{I}_{1}}{λ} ) ] & ( XII ) \\ \frac{\partial pV}{\partial {I}_{1}} = \frac{2 \cdot {e}^{{β}^{′} κ} \cdot cosh ( 2 {β}^{′} ( {I}_{1}^{′} - 3 ) )}{{e}^{{β}^{′} κ} \cdot cosh ( 2 {β}^{′} ( {I}_{1}^{′} - 3 ) ) + 1} & ( XIII )\end{matrix}$$

Wherein n represents a number of links between the cross-linked points in the statistical molecule chain, κ expresses an intermolecular interaction energy coefficient, ν represents a cross-link density and λ represents an extension ratio or compression ratio. β′=1/R(T−T_{g}) wherein R is a gas constant and T_{g} is a glass transition temperature. The term n is also a function of ΔU through X_{c}, and so needs to be differentiated with respect to I₁, however since the contribution is small the integrated term is ignored. I₂ is represented by I₂=λ₁²·λ₂²+λ₂²·λ₃²+λ₃²·λ₁². I₁′ is expressed using a local interaction function λ_{micro} as a parameter expressing the intermolecular interaction by following Equation (XIV):

I₁′=λ_{micro}²(λ₁²+λ₂²+λ₃²)=λ_{micro}²·I₁ (XIV)

According to an eleventh aspect of the present invention, the elastic response performance expressing deformation behavior of the rubber product may be predicted using a finite element analysis method.

In the present invention as described in detail above, the elastic response performance of a rubber product is predicted employing a constitutive equation that expresses temperature and strain dependence of strain energy in a rubber product, or by employing a constitutive equation that expresses temperature and strain dependence of an elastic modulus of the rubber product. It is accordingly possible through necessary simulation, and in particular through simulation at the micro-level of rubber, to design an optimum rubber material at the micro-level to achieve desired characteristics of a rubber product. A design method for rubber products that has both good efficiency and precision can also be provided.

### Advantageous Effects of Invention

As explained above, according to the elastic response performance prediction method, the rubber product design method and the elastic response performance prediction apparatus of the present invention, an elastic response performance of a rubber product is predicted by employing a constitutive equation that expresses temperature and strain dependence of strain energy in the rubber product, and that incorporates a number of links between cross-linked points in a statistical molecule chain which is expressed using a parameter representing extension crystallization, or by employing a constitutive equation that expresses temperature and strain dependence of an elastic modulus, and that incorporates a number of links between cross-linked points in a statistical molecule chain which is expressed using a parameter representing extension crystallization. An advantageous effect is accordingly achieved of enabling the elastic response performance of the rubber product to be predicted with good precision even when the rubber product exhibits extension crystallization properties.

### BRIEF DESCRIPTION OF DRAWINGS

FIG. 1 is a schematic diagram illustrating an elastic response performance prediction apparatus according to an exemplary embodiment of the present invention.

FIG. 2 is an image of a microstructure model.

FIG. 3 is a graph illustrating prediction values and observed values related to the temperature dependence of a stress-strain curve of cross-linked styrene butadiene rubber.

FIG. 4 is a graph illustrating measurement values and computed values related to stress due to energy elasticity and stress due to entropy elasticity of a stress-strain curve at room temperature for a cross-linked styrene butadiene rubber.

FIG. 5 is a graph illustrating measurement values and computed values related to stress due to energy elasticity and stress due to entropy elasticity of a stress-strain curve at room temperature for a cross-linked natural rubber.

FIG. 6 is a graph illustrating prediction values related to temperature dependence of a stress-strain curve for rubber using related technology and observed values.

### BEST MODE FOR CARRYING OUT THE INVENTION

Detailed explanation follows regarding an exemplary embodiment of the present invention, with reference to the drawings.

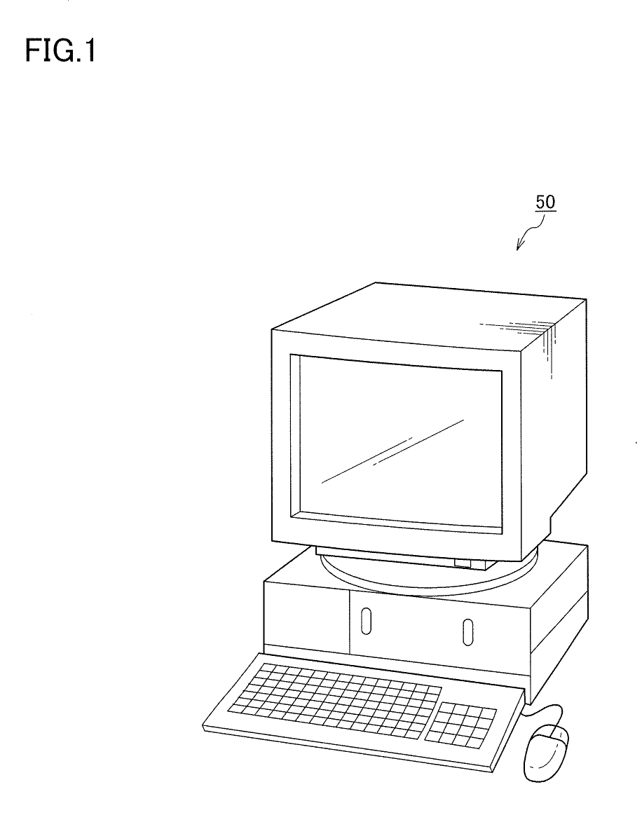

As illustrated in FIG. 1, an elastic response performance prediction apparatus 50 according to a first exemplary embodiment of the present invention is configured by a computer computation processing system that executes processing, described later, with an elastic response performance prediction program in order to execute elastic response performance prediction. Note that such a computer system is, for example, configured with a CPU, ROM, RAM, a hard disk, an input-output terminal, and other required units. The elastic response performance prediction program referred to above is preinstalled on a hard disk or the like.

The elastic response performance prediction method of the first exemplary embodiment of the present invention employs a constitutive equation expressing temperature and strain dependence of strain energy in a rubber material constituting a rubber product, and in particular preferably employs the Equations (I) to (VII) described above, to predict the elastic response performance of the rubber product.

Explanation follows regarding derivation principles of a constitutive equation expressing the temperature and strain dependence of strain energy in a rubber material. In the following, a microstructure model is defined for statistical thermodynamic computation, and a constitutive equation is derived based on this model.

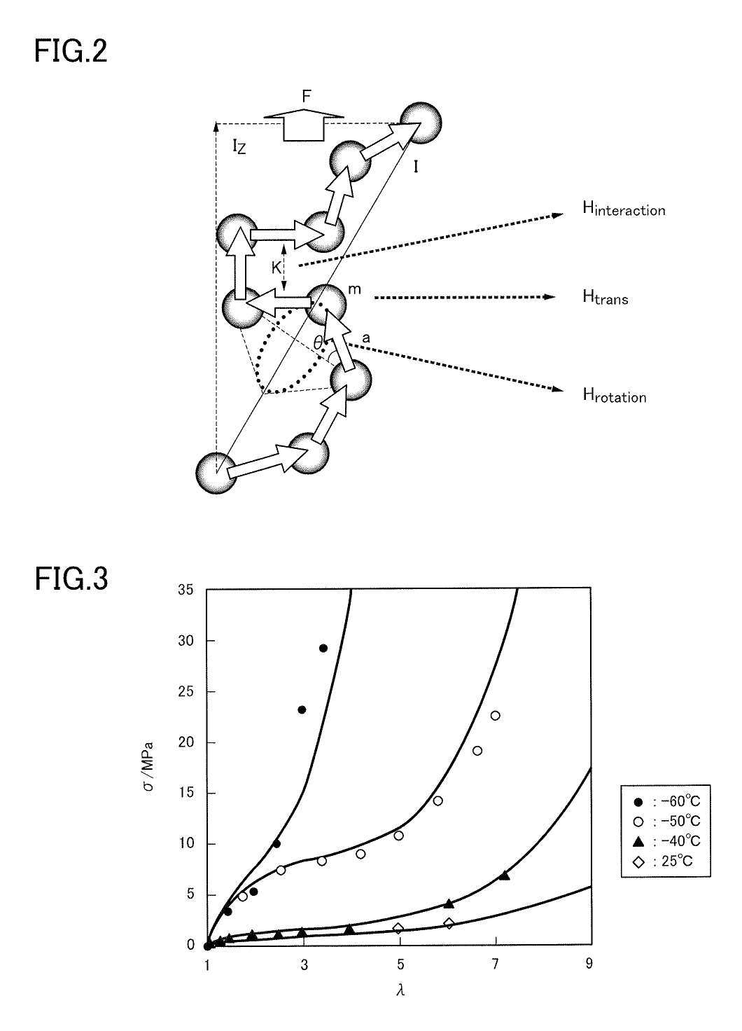

FIG. 2 illustrates a microstructure model. Hᵢₙₜ is a Hamiltonian expressing intermolecular or intramolecular interaction, Hᵣₒₜ is a Hamiltonian expressing molecular rotation, and Hₜᵣₐₙₛ is a Hamiltonian expressing molecular translation.

A thermodynamic equation representing this microstructure model can be expressed by the following Equation (11).

ΔA=ΔU+pΔV−TΔS=∫( Z̅ᵢₙₜ, Z̅ᵣₒₜ, Z̅ₜᵣₐₙₛ)=g(Hᵢₙₜ,Hᵣₒₜ,Hₜᵣₐₙₛ) (11)

Wherein: A represents Helmholz free energy, U represents internal energy, and p represents pressure. V represents volume, T represents absolute temperature and S represents entropy. Z̅ᵢₙₜ represents a distribution function expressing intermolecular or intramolecular interaction, Z̅ᵣₒₜ represents a distribution function expressing molecular rotation, and Z̅ₜᵣₐₙₛ represents a distribution function expressing molecular translation. The Hamiltonians corresponding to each of the molecular motion modes are expressed by the following Equation (12) to Equation (14).

$$\begin{matrix}{H}_{interaction} = ( {r}_{1} + {r}_{2} ) \cdot I - κ \cdot {δ}_{{r}_{1} , {r}_{2}} & ( 12 ) \\ {H}_{trans} = \frac{{p}^{2}}{2 \cdot m} + ( {r}_{1} + {r}_{2} ) \cdot I - κ \cdot {δ}_{{r}_{1} , {r}_{2}} & ( 13 ) \\ {H}_{rotation} = \frac{{p}_{θ}^{2}}{2 \cdot {I}_{M}} + \frac{{p}_{θ}^{2}}{2 \cdot {I}_{M} \cdot {sin}^{2} θ} - F \cdot a \cdot cos θ & ( 14 )\end{matrix}$$

Wherein r₁ and r₂ represent energy states of two molecular with intermolecular or intramolecular interaction. When I₁′ represents an invariant of intermolecular strain in the microstructure model illustrated in FIG. 2, then r₁ and r₂ are expressed by either +1 or −1. r₁ and r₂ corresponds to the four combinations of strain energy due to deformation represented by Iᵢ′, (−1, −1), (+1, +1), (+1, −1), and (-1, +1). κ is a coefficient expressing the magnitude of interaction energy. δ is a coefficient expressing a state involving interaction energy, and is −1 when two individual molecules are in the same energy state. When the two individual molecules are in the same energy state, κ·δ becomes a minus coefficient, meaning stabilization by the amount of the interaction energy. However, when in different energy states, κ·δ becomes 0, assuming that no interaction occurs. m expresses molecular mass, P_{θ} represents momentum expressing an angle of molecular rotation with respect to an axis connecting two molecules together. P_{φ} represents momentum expressing molecular rotation with respect to a rotation angle around the axis connecting two molecules together, P represents momentum with respect to a molecule of mass m, and I_{M} an inertial moment of molecular rotation.

A relationship between a Hamiltonian and a distribution function is expressed by the following Equation (15).

z_{α}=Σ{e^{β·H}^{_{α}}} (15)

Substituting Hamiltonians corresponding to each of the molecule motion modes into the relationship equation expressed above by Equation (15) gives each of the distribution functions expressed by the following Equation (16) to Equation (18).

$$\begin{matrix}{z}_{int} = \sum \{ {e}^{{β}^{′} \cdot ( - {I}_{1}^{′} - {I}_{1}^{′} - κ )} + {e}^{{β}^{′} \cdot ( - {I}_{1}^{′} - {I}_{1}^{′} )} + {e}^{{β}^{′} \cdot ( {I}_{1}^{′} - {I}_{1}^{′} )} + {e}^{{β}^{′} \cdot ( {I}_{1}^{′} - {I}_{1}^{′} - κ )} \} & ( 16 ) \\ {z}_{rot} = \sum exp \{ β \cdot ( \frac{{P}_{θ}^{2}}{2 \cdot {I}_{M}} + \frac{{P}_{ϕ}^{2}}{2 \cdot {I}_{M} \cdot {sin}^{2} θ} - F \cdot a \cdot cos θ ) \} & ( 17 ) \\ {z}_{trans} = \sum exp \{ β \cdot ( \frac{{P}^{2}}{2 m} ) \} + \sum \{ {e}^{{β}^{′} \cdot ( - {I}_{1}^{′} - {I}_{1}^{′} - κ )} + {e}^{{β}^{′} \cdot ( - {I}_{1}^{′} - {I}_{1}^{′} )} + {e}^{{β}^{′} \cdot ( {I}_{1}^{′} - {I}_{1}^{′} )} + {e}^{{β}^{′} \cdot ( {I}_{1}^{′} - {I}_{1}^{′} - κ )} \} & ( 18 )\end{matrix}$$

Solving the above relationships derives the following Equation (19) to Equation (21).

$$\begin{matrix}{z}_{int} = 2 [ {e}^{{β}^{′} κ} cosh \{ 2 {β}^{′} ( {I}_{1}^{′} - 3 ) \} + 1 ] & ( 19 ) \\ {z}_{rot} = \frac{2 \cdot {I}_{M}}{β} \cdot \frac{sinh ( β \cdot F \cdot a )}{β \cdot F \cdot a} & ( 20 ) \\ {z}_{trans} = exp \{ λ \cdot Λ \cdot {( \frac{2 π \cdot m}{β} )}^{\frac{3}{2}} \} + 2 [ {e}^{{β}^{′} κ} cosh \{ 2 {β}^{′} ( {I}_{1}^{′} - 3 ) \} + 1 ] & ( 21 )\end{matrix}$$

These distribution functions are for a localized model as illustrated in FIG. 2, and the following Equation (22) to Equation (24) are obtained when expanded to a macro-model with N molecules.

$$\begin{matrix}{\overline{Z}}_{int} = \frac{{z}_{int}^{N}}{N !} & ( 22 ) \\ {\overline{Z}}_{rot} = \frac{{z}_{rot}^{N}}{N !} & ( 23 ) \\ {\overline{Z}}_{trans} = \frac{{z}_{trans}^{N}}{N !} & ( 24 )\end{matrix}$$

Wherein: Z̅ᵢₙₜ represents a distribution function of intermolecular or intramolecular interaction expanded to N molecules, Z̅ᵣₒₜ represents a distribution function of molecular rotation expanded to N molecules, Z̅ₜᵣₐₙₛ represents a distribution function of molecular translation expanded to N molecules.

The relationships between the distribution functions and the thermodynamic state functions are expressed by the following Equation (25) to Equation (27).

$$\begin{matrix}U = - {( \frac{\partial ln {\overline{Z}}_{int}}{\partial {β}^{′}} )}_{V} & ( 25 ) \\ \begin{matrix}S = k \cdot ln Ω ( F ) \\ = ln {\overline{Z}}_{rot} - β \cdot F \cdot a\end{matrix} & ( 26 ) \\ pV = \frac{1}{β} \cdot ln {\overline{Z}}_{trans} & ( 27 )\end{matrix}$$

Wherein β represents 1/RT. When T_{g} is the glass transition temperature, then β′ is expressed by:

$$\frac{1}{R ( T - {T}_{s} )}$$

n represents a number of links between cross-link points in a statistical molecule chain, and E represents an intermolecular interaction energy coefficient. Ω(F) represents a number of states adopted by molecules, with F representing the force generated by a deformed molecule, a represents a statistical molecule length, and k representing the Boltzmann constant. The above Equation (III) is derived by substituting a distribution function for N molecules into these equations. Consequently, a change in Helmholz free energy due to deformation in rubber is expressed as a difference between a non-deformed state and a deformed state.

Each of the terms in above Equation (III) correspond to the respective Equations (IV) to (VI) above. Wherein, I₁′ considers a local interaction function λ_{micro} and is defined by above Equation (VII).

In the elastic response performance prediction method of the present exemplary embodiment, the elastic response performance of a rubber product is predicted by employing a constitutive equation expressed by the Equation (III) to Equation (VII) derived as described above.

Moreover, in the present exemplary embodiment, the number of links n between cross-linked points in a statistical molecule chain is defined by the above Equation (I). X_{c} represents the crystallization ratio, and is represented by above Equation (II) as a function of internal energy change ΔU=U₁−U₀ only in cases in which the subject material exhibits extension crystallization properties.

Wherein, ΔH when crystals melt is equivalent to the entropy of solution, and 6MJ/m³ is employed as ΔH in cases of for natural rubber. Note that X_{c} is taken as 0 in cases in which the subject material does not exhibit extension crystallization properties. Moreover, X_{c} is never 1 or greater, since there can never be more than 100% crystallization due to extension.

In the a constitutive equation described above, appropriate application of the above Equation (I) and Equation (II) enables consideration to be made of the temperature and stress dependence even in cases in which stress-strain behavior analysis technology is applied at a micro-level in rubber with extension crystallization properties.

Moreover, by appropriate application of Equation (I) and Equation (II) to the Equations (III), (IV) and (VI), an internal energy term U is expressed as a negative value (exothermic) the larger the deformation of rubber with extension crystallization properties. Moreover, in a stretched molecule phenomenon there are limitations to the entropy S by molecular mobility between cross-linked points due to crystallization, and the value of n becomes smaller, and S increases, in proportion to the crystallization ratio. A constitutive equation is accordingly obtained expressing stretched molecules or non-linear behavior in regions where large deformation is observed due to extension crystallization.

Explanation follows regarding operation when prediction of deformation behavior in a rubber material is performed according to the first exemplary embodiment.

First, a 3D-model of a rubber product, such as a tire, is generated by the computer 50, and the 3D-model data is stored on the HDD.

Next, a user operates the input-output terminal to set the 3D-model to be subjected to analysis. The user then operates the input-output terminal to set analysis conditions for analysis processing. The analysis condition setting includes framework conditions of the rubber portion of the 3D-model, and setting framework conditions for filler portions. As framework conditions of the rubber portion of the 3D-model, the constitutive equation of the above Equations (I) to (VII) is set (when the rubber exhibits extension crystallization), or the constitutive equation of Equations (I) and (III) to (VII) is set (when the rubber does not exhibit extension crystallization).

The computer 50 then reads in the 3D-model data of the 3D-model set as the subject of analysis from the HDD, and applies the set analysis conditions as the framework conditions of the rubber portion and the filler portion of the 3D-model expressed by the read-in 3D-model data, so as to reconstruct the 3D-model data.

Next, the computer 50 employs the reconstructed 3D-model data to analyze with a finite element method the strain and the internal stress distribution in the 3D-model, and the stress values of the 3D-model as a whole resulting from vehicle load bearing on the tire, internal pressure of the tire, rolling of the tire etc. when the 3D-model is changed under the framework conditions that have been set.

The computer 50 then displays on a display the strain state and the internal stress distribution of the 3D-model, and the stress values of the 3D-model as a whole that have been derived by analysis, and then ends processing.

Then the constitutive equation described above, this being the Equations (I) and (III) to (VII), are employed and prediction of the temperature dependence of the stress-strain curve of the rubber is performed, and the temperature dependence of the stress-strain curve is also measured using the cross-linked styrene butadiene rubber. A comparison is then performed between the prediction values (solid line) by the prediction described above, and the measurement values (plots). A comparison result is illustrated in FIG. 3. It can be said that there is a good match shown between the curves obtained with the Equations (I) and (III) to (VII), particularly in low extension ratio regions, and the measurement value plots.

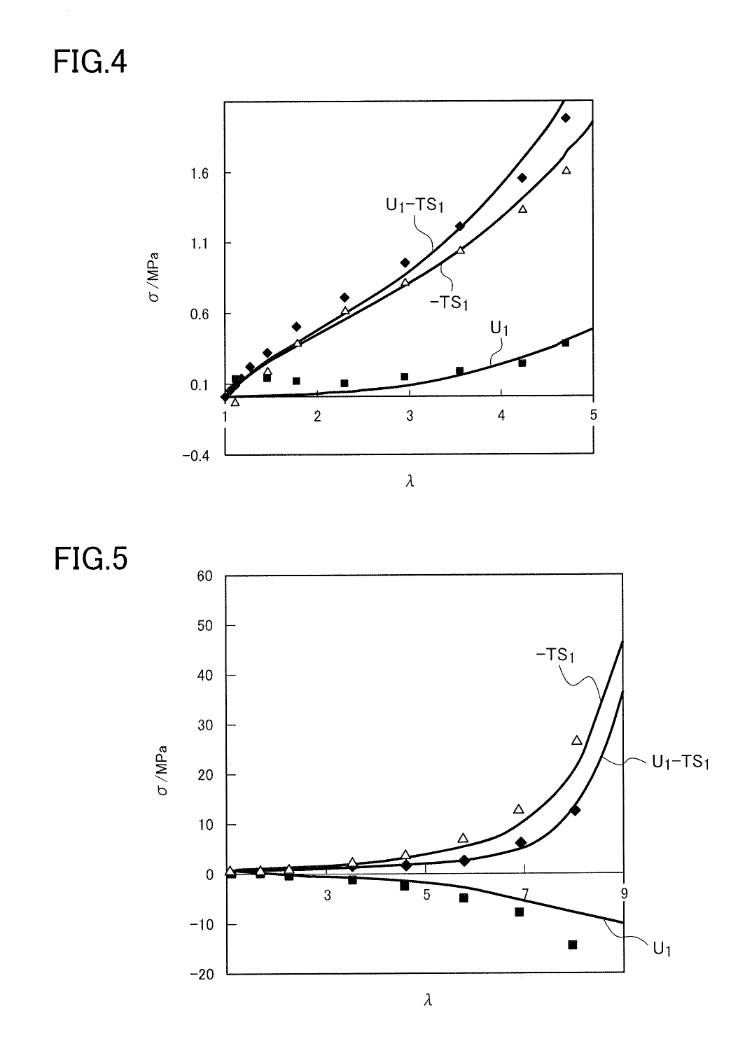

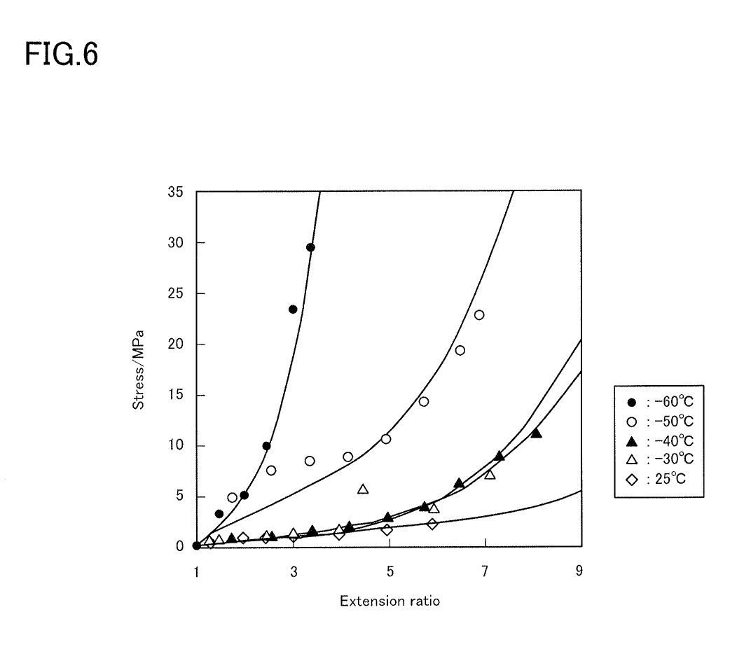

Moreover, cross-linked styrene butadiene rubber is employed, measurement of the stress due to energy elasticity and the stress due to entropy elasticity for a stress-strain curve at room temperature is performed, and computation is also performed of the corresponded portion (U₁, −TS₁) of the Equations (III), (IV) in the above constitutive equation. A comparison is performed between the above measurement value (plots) and computed values (solid lines). Comparison results are illustrated in FIG. 4. It can be seen that there is a good match between the curves obtained by the corresponded portion of the Equations (III), (IV) and the plots of the measurement values.

Moreover cross-linked natural rubber is employed, measurement is performed of the stress due to energy elasticity and the stress due to entropy elasticity for a stress-strain curve at room temperature, and the Equations (I) and (II) are also applied and computation performed of the corresponded portion (U₁, −TS₁) of the Equations (III), (IV) in the above constitutive equation. A comparison is made between the above measurement values (plots) and computed values (solid lines). Comparison results are illustrated in FIG. 5. It can be seen that there is a good match between the curves obtained by the corresponded portion of the Equations (III), (IV) and the plots of the measurement values. Since natural rubber exhibits extension crystallization, there is a tendency for the energy elasticity to become minus when the extension ratio is 3 or greater. Since such a phenomenon is also well represented, it can be said that good modeling can also be achieved even for extension crystallization.

As explained above, according to a rubber product elastic response performance prediction apparatus according to the first exemplary embodiment, a crystallization ratio expressing the extension crystallization at high strain in a rubber component exhibiting extension crystallization properties, such as NR, is incorporated into a constitutive equation expressing temperature and strain dependence of strain energy, so as to predict the elastic response performance of a rubber product. The elastic response performance can accordingly be predicted with good precision even if the rubber product exhibits extension crystallization properties.

Moreover, when a filler based rubber is observed at the micro-observation level, in a diffuse deformation region of a rubber product, a constitutive equation is employed that expresses temperature and strain dependence of strain energy employing a local interaction function λ_{micro} incorporated with the effects of adjacent molecules at low strain, so as to predict the elastic response performance of a rubber product. Accordingly, elastic response performance in low temperature conditions, particularly in a low extension ratio region, can be predicted with good precision.

A microstructure model is defined, and errors in portions of interaction as viewed in micro, in particular extension crystallization properties of natural rubber in high deformation regions, are incorporated, enabling the divergence from observed values to be reduced. The elastic response performance of a rubber product containing natural rubber can accordingly be predicted with good precision.

Moreover, a feature is that the energy elasticity is considered in addition to conventional consideration of the contribution of entropy elasticity representing rubber elasticity. The energy elasticity is considered in a similar manner to the strain energy function proposed in Japanese Patent No. 4299735, however a microstructure model thereof was not clearly defined. Hence errors in the portions of interaction as viewed in micro, and in particular the contribution of energy elasticity in low deformation regions, and the phenomenon called extension crystallization where unable to be represented. In the present invention there is a clear microstructure model for statistical thermodynamic computation, and by deriving a constitutive equation based on this model, a universal constitutive equation for rubber elasticity is derived that does not depend on the deformation region or the type of rubber.

With a conventional rubber constitutive equation it was not possible to express the thermodynamic behavior of the rubber, as is illustrated in FIG. 4 and FIG. 5. In the present invention, a constitutive equation expressing the thermodynamic behavior of rubber such as illustrated in FIG. 4 and FIG. 5 is introduced into a conventional computational simulation. It is accordingly possible to perform predictive computation in a state reflecting the thermodynamic behavior of raw materials actually employed.

Moreover, the temperature dependence of a stress-strain curve expressed by employing the constitutive equation obtained from statistical thermodynamic computation has been confirmed to have a good match with results from measurements including those for natural rubber that exhibits extension crystallization properties. Thus by introducing the proposed constitutive equation into a finite element method stress-strain curve, it is possible to represent the rubber stress-strain behavior of rubber at the nanoscale for both rubbers with extension crystallization properties and rubbers without crystallization properties, including elasticity contributions, viscosity contributions, and plasticity contributions therein.

Explanation next follows regarding a second exemplary embodiment.

The second exemplary embodiment differs from the first exemplary embodiment regarding the prediction of elastic response performance of a rubber product in the point that a constitutive equation is employed that expresses the temperature and strain dependence of an elastic modulus of a rubber product.

In an elastic response performance prediction method of the second exemplary embodiment a constitutive equation expressed by above Equation (X), which is derived similarly to that of the first exemplary embodiment, is applied to a deformation behavior analysis technique for rubber at the micro-level to predict the elastic response performance of a rubber product.

Each of the terms in the above Equation (X) are expressed by the above Equations (XI), (XII) and (XIII). Wherein, I₁′ is expressed by employing a local interaction function λ_{micro} and is expressed by Equation (XIV).

In the present exemplary embodiment, the number of links n between cross-linked points in a statistical molecule chain is defined using the above Equations (VIII) and (IX), so as to represent extension crystallization. Thus temperature and strain dependence can be considered even in cases in which a deformation behavior analysis technique is applied at the micro-level to a rubber with extension crystallization properties.

Note that since other configuration and operation of the elastic response performance prediction apparatus according to a second exemplary embodiment of the present invention are similar to those of the first exemplary embodiment, explanation is omitted thereof.

Thus according to the rubber product elastic response performance prediction apparatus according to the second exemplary embodiment, a crystallization ratio expressing extension crystallization at high strain in a rubber component exhibiting extension crystallization properties, such as NR, is incorporated into a constitutive equation expressing the temperature and strain dependence of an elastic modulus, so as to predict the elastic response performance of a rubber product. The elastic response performance can accordingly be predicted with good precision even if the rubber product exhibits extension crystallization properties.

Moreover, when a filler based rubber is observed at the micro-observation level, in a diffuse deformation region of a rubber product, a constitutive equation is employed that expresses temperature and strain dependence of an elastic modulus employing a local interaction function λ_{micro} incorporated with the effects of adjacent molecules at low strain, so as to predict the elastic response performance of a rubber product. Accordingly, elastic response performance in low temperature conditions, particularly in a low extension ratio region, can be predicted with good precision.

A microstructure model is defined, and errors in portions of interaction as viewed in micro, in particular extension crystallization properties of natural rubber in high deformation regions, are incorporated, enabling the divergence from observed values to be reduced. The elastic response performance of a rubber product containing natural rubber can accordingly be predicted with good precision.

The entire content disclosed in Japanese Patent Application No. 2010-225984 is incorporated by reference into the present specification.

All cited documents, patent applications and technical standards mentioned in the present specification are incorporated by reference in the present specification to the same extent as if the individual cited document, patent application, or technical standard was specifically and individually indicated to be incorporated by reference.

1. An elastic response performance prediction method that predicts an elastic response performance expressing deformation behavior of a rubber product, the elastic response performance prediction method comprising predicting the elastic response performance of the rubber product by employing a constitutive equation that expresses temperature and strain dependence of strain energy in the rubber product, and that incorporates a number of links between cross-linked points in a statistical molecule chain which is expressed using a parameter representing extension crystallization.

2. The elastic response performance prediction method of claim 1, wherein:
a number of links n between the cross-linked points in the statistical molecule chain is expressed by the following Equation (I):
n=α·(1−X_{c})·exp(−ε·β) (I)
wherein α represents a frequency factor of statistical segment motion, ε represents an activation energy of statistical segment motion, β=1/RT_{g} wherein R is a gas constant and T_{g} is a glass transition temperature, X_{c} represents a crystallization ratio as a parameter expressing the extension crystallization, and X_{c} is expressed by the following Equation (II) when a material of the rubber product exhibits extension crystallization properties:
$$\begin{matrix}{X}_{c} = ( \frac{{U}_{1} - {U}_{0}}{Δ {H}_{0}} ) \\ = ( \frac{Δ U}{Δ {H}_{0}} )\end{matrix} \qquad ( II )$$
wherein U₀ represents internal energy in a non-deformed state, U₁ represents internal energy in a deformed state, and ΔH₀ represents entropy of solution when crystals melt.

3. The elastic response performance prediction method of claim 2, wherein the crystallization ratio X_{c} is set at 0 when the material of the rubber product does not exhibit extension crystallization properties.

4. The elastic response performance prediction method of claim 1, wherein the constitutive equation is the following Equation (III):
ΔA=(U₁−TS₁)+p(V₁−V₀)−(U₀−TS₀) (III)
wherein A represents Helmholz free energy, U₀ represents internal energy in a non-deformed state, U₁ represents internal energy in a deformed state, p represents pressure, V₀ represents volume in a non-deformed state, V₁ represents volume in a deformed state, T represents absolute temperature, S₀ represents entropy in a non-deformed state, and S₁ represents entropy in a deformed state, with each of the terms of Equation (III) expressed by the following Equations (IV) to (VI):
$$\begin{matrix}{U}_{1} - T \cdot {S}_{1} = \frac{{e}^{{β}^{′} \cdot κ} \{ κ cosh ( 2 {β}^{′} ( {I}_{1}^{′} - 3 ) ) + 2 ( {I}_{1}^{′} - 3 ) \cdot sinh ( 2 {β}^{′} ( {I}_{1}^{′} - 3 ) ) \}}{{e}^{{β}^{′} \cdot κ} cosh ( 2 {β}^{′} ( {I}_{1}^{′} - 3 ) ) + 1} - \frac{N}{β} \cdot \{ \frac{1}{2} {I}_{1} + \frac{3}{100 n} ( 3 {I}_{1}^{2} - 4 {I}_{2} ) + \frac{99}{12250 n} ( 5 {I}_{1}^{3} - 12 {I}_{1} {I}_{2} ) \} & ( IV ) \\ \begin{matrix}p ( {V}_{1} - {V}_{0} ) = B \cdot ( {V}_{1} - {V}_{0} ) \\ = {B ( {I}_{3}^{\frac{1}{2}} - 1 )}^{2} - \frac{1}{{β}^{′}} \{ \begin{matrix}ln [ 1 + {e}^{{β}^{′} \cdot κ} cosh ( 2 {β}^{′} ( {I}_{1}^{′} - 3 ) ) ] - \\ ln [ 1 + {e}^{{β}^{′} \cdot κ} ]\end{matrix} \}\end{matrix} & ( V ) \\ {U}_{0} - T \cdot {S}_{0} = \frac{κ \cdot {e}^{{β}^{′} \cdot κ}}{{e}^{{β}^{′} \cdot κ} + 1} + \frac{N}{β} ( \frac{3}{2} + \frac{45}{100 n} + \frac{2673}{12250 {n}^{2}} ) & ( VI )\end{matrix}$$
wherein: I₁, I₂, and I₃ are expressed as functions of three extension ratios of deformation λ₁, λ₂ and λ₃ in xyz directions in three dimensional axes of rubber by I₁=λ₁²+λ₂²+λ₃², I₂=λ₁²·λ₂²+λ₂²·λ₃²+λ₃²·λ₁², and I₃=λ₁²·λ₂²·λ₃², n represents the number of links between the cross-linked points in the statistical molecule chain, κ expresses an intermolecular interaction energy coefficient, β=1/RT and β′=1/R(T−T_{g}) wherein R is a gas constant and T_{g} is a glass transition temperature, and I₁′ is expressed using a local interaction function λ_{micro} as a parameter expressing the intermolecular interaction by the following Equation (VII):
I₁′=λ_{micro}²(λ₁²+λ₂²+λ₃²)=λ_{micro}²·I₁ (VII)

5. An elastic response performance prediction method that predicts elastic response performance expressing deformation behavior of a rubber product, the elastic response performance prediction method comprising predicting the elastic response performance of the rubber product by employing a constitutive equation that expresses temperature and strain dependence of an elastic modulus of the rubber product, and that incorporates a number of links between cross-linked points in a statistical molecule chain which is expressed using a parameter representing extension crystallization.

6. The elastic response performance prediction method of claim 5, wherein the number of links n between the cross-linked points in the statistical molecule chain is expressed by the following Equation (VIII):
n=α·(1−X_{c})·exp(−ε·β) (VIII)
wherein α represents a frequency factor of statistical segment motion, ε represents an activation energy of statistical segment motion, β=1/RT_{g} wherein R is a gas constant and T_{g} is a glass transition temperature, X_{c} represents a crystallization ratio as a parameter expressing the extension crystallization, and X_{c} is expressed by the following Equation (IX) when a material of the rubber product exhibits extension crystallization properties:
$$\begin{matrix}{X}_{c} = ( \frac{{U}_{1} - {U}_{0}}{Δ {H}_{0}} ) \\ = ( \frac{Δ U}{Δ {H}_{0}} )\end{matrix} \qquad ( IX )$$
wherein U₀ represents internal energy in a non-deformed state, U₁ represents internal energy in a deformed state, and ΔH₀ represents entropy of solution when crystals melt.

7. The elastic response performance prediction method of claim 6, wherein the crystallization ratio X_{c} is set at 0 when the material of the rubber product does not exhibit extension crystallization properties.

8. The elastic response performance prediction method of claim 5, wherein the constitutive equation is the following Equation (X):
$$\begin{matrix}G = \frac{\partial W}{\partial {I}_{1}} \\ = \frac{\partial A}{\partial {I}_{1}} \\ = \frac{\partial U}{\partial {I}_{1}} - T \cdot \frac{\partial S}{\partial {I}_{1}} + \frac{\partial pV}{\partial {I}_{1}}\end{matrix} \qquad ( X )$$
wherein G represents a shear elastic modulus, W represents a strain energy coefficient, A represents Helmholz free energy, U represents internal energy, T represents absolute temperature, S represents entropy, and I₁ is expressed as a function of three extension ratios of deformation λ₁, λ₂ and λ₃ in xyz directions in three dimensional axes of rubber by I₁=λ₁²+λ₂²+λ₃², with each of the terms of Equation (X) respectively expressed by the following Equation (XI), Equation (XII), and Equation (XIII):
$$\begin{matrix}\frac{\partial U}{\partial {I}_{1}} = \frac{\begin{matrix} \\ - {e}^{{β}^{′} κ} \{ \begin{matrix}\begin{matrix}2 {e}^{{β}^{′} κ} sinh ( 2 {β}^{′} ( {I}_{1}^{′} - 3 ) ) cosh ( 2 {β}^{′} ( {I}_{1}^{′} - 3 ) ) + \\ 2 ( {β}^{′} κ + 1 ) sinh ( 2 {β}^{′} ( {I}_{1}^{′} - 3 ) ) +\end{matrix} \\ 4 {β}^{′} ( {I}_{1}^{′} - 3 ) cosh ( 2 {β}^{′} ( {I}_{1}^{′} - 3 ) ) + \\ 4 {β}^{′} ( {I}_{1}^{′} - 3 ) {e}^{{β}^{′} κ}\end{matrix} \}\end{matrix}}{{\{ {e}^{{β}^{′} κ} \cdot cosh ( 2 {β}^{′} ( {I}_{1}^{′} - 3 ) ) + 1 \}}^{2}} & ( XI ) \\ \frac{\partial S}{\partial {I}_{1}} = - vR [ \frac{1}{2} + \frac{3}{50 n} ( 3 {I}_{1} - \frac{2}{λ} ) + \frac{297}{6125 {n}^{2}} ( 5 {I}_{1}^{2} - 4 {I}_{2} - 4 \frac{{I}_{1}}{λ} ) ] & ( XII ) \\ \frac{\partial pV}{\partial {I}_{1}} = \frac{2 \cdot {e}^{{β}^{′} κ} \cdot cosh ( 2 {β}^{′} ( {I}_{1}^{′} - 3 ) )}{{e}^{{β}^{′} κ} \cdot cosh ( 2 {β}^{′} ( {I}_{1}^{′} - 3 ) ) + 1} & ( XIII )\end{matrix}$$
wherein κ expresses an intermolecular interaction energy coefficient, n represents the number of links between the cross-linked points in the statistical molecule chain, v represents a cross-link density, λ represents an extension ratio or compression ratio, β′=1/R(T−T_{g}) wherein R is a gas constant and T_{g} is a glass transition temperature, I₂ is represented by I₂=λ₁²·λ₂²+λ₂²·λ₃²+λ₃²·λ₁², and I₁′ is expressed using a local interaction function λ_{micro} as a parameter expressing the intermolecular interaction by the following Equation (XIV):
I₁′=λ_{micro}²(λ₁²+λ₂²+λ₃²)=λ_{micro}²·I₁ (XIV)

9. The elastic response performance prediction method of claim 1, wherein the elastic response performance expressing deformation behavior of the rubber product is predicted using a finite element analysis method.

10. A rubber product design method comprising designing a rubber product by employing the elastic response performance prediction method of claim 1.

11. An elastic response performance prediction apparatus that predicts elastic response performance expressing deformation behavior of a rubber product, the elastic response performance prediction apparatus predicting the elastic response performance of the rubber product by employing a constitutive equation that expresses temperature and strain dependence of strain energy in the rubber product, and that incorporates a number of links between cross-linked points in a statistical molecule chain which is expressed using a parameter representing extension crystallization.

12. An elastic response performance prediction apparatus that predicts elastic response performance expressing deformation behavior of a rubber product, the elastic response performance prediction apparatus predicting the elastic response performance of the rubber product by employing a constitutive equation that expresses temperature and strain dependence of an elastic modulus of the rubber product, and that incorporates a number of links between cross-linked points in a statistical molecule chain which is expressed using a parameter representing extension crystallization.
